(19) United States
(12) Patent Application Publication
(10) Pub. No.: US 2025/0359942 A1
(43) Pub. Date: Nov. 27, 2025

(54) SYSTEM FOR MOVING A MEDICAL OBJECT, METHOD FOR PROVIDING A CONTROL SPECIFICATION, AND COMPUTER PROGRAM PRODUCT

(71) Applicant: Siemens Healthineers AG, Forchheim (DE)

(72) Inventors: Markus Kowarschik, Erlangen (DE); Tanja Kurzendorfer, Neumarkt (DE)

(21) Appl. No.: 19/209,784

(22) Filed: May 15, 2025

(30) Foreign Application Priority Data
May 21, 2024 (DE) 10 2024 204 665.5

(51) Int. Cl. A61B 34/20; A61B 34/10

(52) U.S. Cl. CPC A61B 34/20; A61B 34/10; A61B 2034/107

(57) ABSTRACT

A system for moving a medical object includes a processing unit, a movement apparatus, and a capturing unit. The processing unit is configured to capture a preprocedural dataset of an examination object that has a representation of a hollow organ of the examination object having a predefined section of the medical object arranged in the hollow organ. The capturing unit is configured to capture an instantaneous positioning of the predefined section. The processing unit is configured to: identify a curvature of the hollow organ at the instantaneous positioning; and provide the movement apparatus with a control specification relating to the movement of the medical object on the basis of the curvature of the hollow organ, wherein the control specification has a specification relating to the manipulation of the predefined section. The movement apparatus is configured to move in accordance with the control specification and to manipulate the predefined section.

The present patent document claims the benefit of German Patent Application No. 10 2024 204 665.5, filed May 21, 2024, which is hereby incorporated by reference in its entirety.

### TECHNICAL FIELD

The present disclosure relates to a system for moving a medical object, to a method for providing a control specification and to a computer program product.

### BACKGROUND

Endovascular robotics for minimally invasive procedures are becoming increasingly important in various clinical disciplines, for example, in interventional radiology, interventional oncology, and interventional neuroradiology. The advantages of endovascular robotics include a high level of precision, speed, and patient safety. In the context of endovascular robotics, there is a need for the autonomous

navigation of interventional instruments. This means that on the basis of a pre-interventional vascular three-dimensional (3D) dataset, (e.g., a computed tomography angiography (CT angiography, CTA) or a magnetic resonance angiography (MR angiography, MRA)), a robotic system automatically moves the interventional instrument inside the body of the patient from a target position, (e.g., a femoral or radial puncture site), to a specific predefined target object, (e.g., a blood clot or a tumor). Potential advantages of autonomous navigation include procedures of shorter length, reduced risk of complications, and, owing to this, a standardization of endovascular interventions.

Regardless of these robotic developments, controllable or articulatable endovascular instruments are more popular. The term “controllable” refers in this case to a dynamic bending property, (e.g., articulating property), of a tip of the instrument, (e.g., within a bending plane), corresponding to the instantaneous vascular anatomy, with it being possible to use suitable interfaces at a proximal end section of the instrument. In addition, the entire instrument may be rotatable in order to provide that the instrument may move in the appropriate directions along a path through the vessels of the patient. Such endovascular instruments may include microcatheters and/or guide wires.

### SUMMARY AND DESCRIPTION

It is therefore the object of the present disclosure to enable autonomous navigation of, in particular controllable, interventional instruments.

The scope of the present disclosure is defined solely by the appended claims and is not affected to any degree by the statements within this summary. The present embodiments may obviate one or more of the drawbacks or limitations in the related art.

In a first aspect, a system for moving a medical object is disclosed. The system includes a processing unit, a movement apparatus, and a capturing unit. The processing unit is configured to receive a preprocedural dataset of an examination object. Further, the preprocedural dataset has a representation of a hollow organ of the examination object. In an operating state of the system, at least one predefined section of the medical object is arranged in the hollow organ of the examination object. The capturing unit is configured to capture an instantaneous positioning of the predefined section of the medical object. The processing unit is also configured to identify a curvature of the hollow organ at the instantaneous positioning of the predefined section using the preprocedural dataset. Further, the processing unit is configured to provide the movement apparatus with a control specification relating to the movement of the medical object on the basis of the curvature of the hollow organ. The control specification has a specification relating to the manipulation of the predefined section. The movement apparatus is configured to move the medical object in accordance with the control specification and manipulate the predefined section.

The examination object may include a female human or animal patient, a male human or animal patient, an examination phantom, (e.g., a vessel phantom), or a combination thereof. The examination object may have a hollow organ, (e.g., a vessel such as an artery or vein), a vascular malformation, a trachea, esophagus, an intestine, or a combination thereof.

The medical object may be configured, for example, as an, in particular, elongate, surgical and/or diagnostic instrument. In particular, the medical object may be configured, at least in certain sections, to be flexible and/or rigid. The medical object may be configured as a needle, (e.g., puncture needle), a catheter, a microcatheter, an endoscope, a guide wire, or a combination thereof.

Advantageously, the predefined section may be a distal section of the medical object. The predefined section may denote a spatial region of the medical object along its direction of longitudinal extension, for example, a tip or an end section of the medical object. Advantageously, the predefined section of the medical object may be configured so it may be manipulated, (e.g., controlled and/or articulated and/or so it may curve and/or bend and/or rotate). In particular, the manipulation of the predefined section may be controllable by a manipulation interface at a proximal section of the medical object. Advantageously, in the operating state of the system, the medical object may be arranged partially in the hollow organ of the examination object. The predefined section of the medical object may be arranged in the hollow organ in the operating state of the system.

Advantageously, the movement apparatus may be a robotic apparatus configured for remote manipulation of the medical object, in particular of the predefined section, for example, a catheter robot and/or an endovascular robot system. Advantageously, the movement apparatus may be arranged outside of the examination object in an operating state of the system. The movement apparatus may advantageously be configured to accommodate the proximal section of the medical object in the operating state of the system. In the operating state of the system, the proximal section may face the movement apparatus, in particular be remote from the examination object. Further, in the operating state of the system, the distal section may be remote from the movement apparatus, in particular face the examination object. In particular, in the operating state of the system, the distal section may be arranged in the hollow organ of the examination object. Further, the movement apparatus may be configured to hold and/or move the medical object by transferring a force and/or energy. In particular, the movement apparatus may be configured to manipulate, in particular arrange and/or move, the predefined section of the medical object by transferring the force and/or energy to the proximal section. In particular, the movement apparatus may have a control interface which is configured to couple to the manipulation interface of the medical object. Advantageously, the force and/or energy may be transferred by the coupled interfaces. Alternatively, or in addition, the control interface may be configured to provide the manipulation interface of the medical object with manipulation specifications.

The capturing unit may have a sensor, (e.g., electromagnetic and/or optical and/or acoustic and/or a mechanical sensor), for capturing the instantaneous positioning of the predefined section. The instantaneous positioning of the predefined section may include an instantaneous spatial position and/or orientation and/or pose of the predefined section. Advantageously, the capturing unit may be configured to capture the instantaneous positioning of the predefined section in the operating state of the system, for example, in respect of the examination object, in particular of the hollow organ. In particular, the capturing unit may be configured to provide the processing unit with an item of positioning information, having an item of information relating to the instantaneous positioning of the predefined section.

Receiving the preprocedural dataset may include capturing and/or reading a computer-readable data memory and/or receiving from a data memory unit, for example, a database. Further, the preprocedural dataset may be provided by a processing unit of a medical imaging device for recording the preprocedural dataset. The medical imaging device may include a magnetic resonance tomography unit (MRT) and/or a computed tomography unit (CT) and/or a medical X-ray device, in particular a medical C-arm X-ray device, and/or an ultrasound device and/or a positron emission tomography unit (PET).

The preprocedural dataset may advantageously include a, in particular time-resolved, two-dimensional (2D) and/or three-dimensional (3D) mapping of the examination object, in particular of the hollow organ of the examination object. In particular, the preprocedural dataset may include a contrasted and/or segmented mapping of the examination object, in particular of the hollow organ. Further, the preprocedural dataset may map the examination object, in particular the hollow organ, pre-procedurally, in particular before arrangement of the predefined section in the hollow organ. Alternatively, or in addition, the preprocedural dataset may have a 2D and/or 3D model, in particular a centerline model and/or a volume model, for example, a volume mesh model, of the examination object, in particular of the hollow organ. The preprocedural dataset may advantageously be registered with a coordinate system of the examination object, in particular a patient coordinate system.

The processing unit is configured to identify the curvature of the hollow organ at the instantaneous positioning of the predefined section using the preprocedural dataset. Advantageously, the processing unit may be configured to register the item of positioning information with the preprocedural dataset. Advantageously, the processing unit may be configured to identify a spatial characteristic of the hollow organ, in particular a surrounding wall of the hollow organ, for example, a vessel wall, and/or a centerline of the hollow organ, using the instantaneous positioning of the predefined section and the preprocedural dataset. The curvature of the hollow organ may be characterized by a deviation of the identified spatial characteristic with respect to a straight-line characteristic and/or a curvature parameter, for example, a radius of curvature and/or a direction of curvature and/or an angle of curvature. Advantageously, the processing unit may be configured to identify the instantaneous curvature of the hollow organ as the instantaneous curvature of a spatial section of the hollow organ along its direction of longitudinal extension at the instantaneous positioning of the predefined section.

Advantageously, the processing unit may be configured to provide the movement apparatus with the control specification relating to the movement of the medical object, in particular in real time, on the basis of the identified curvature of the hollow organ. The control specification may include at least one command for control, in particular incremental and/or continuous control, of the movement apparatus. Advantageously, the control specification may have at least one command, in particular a chronological sequence of commands, relating to the specification of a manipulation, for example, deforming and/or curving and/or bending and/or rotating, of the predefined section. In addition, the control specification may include at least one command, in particular a chronological sequence of commands, relating to the specification of a, in particular simultaneous, translation and/or rotation of the medical object, in particular of the predefined section, by the movement apparatus.

Furthermore, providing the movement apparatus with the control specification may include storage on a computer-readable storage medium and/or display on the display unit and/or transfer to the movement apparatus. Advantageously, the processing unit may be configured to transmit the control specification and to control the movement apparatus on the basis of it. In addition, the movement apparatus may be configured to manipulate, in particular deform and/or curve and/or bend and/or rotate, the predefined section on the basis of the control specification. Furthermore, the movement apparatus may be configured to move the medical object, in particular in a translational and/or rotational manner, on the basis of the control specification.

The proposed system may advantageously enable autonomous, in particular robotic, navigation of, in particular controllable, interventional instruments.

In a further advantageous embodiment of the proposed system, the capturing unit may have a medical imaging device which is configured to record intraprocedural image data of the examination object. The capturing unit may be configured to identify the instantaneous positioning of the predefined section using the intraprocedural image data.

The medical imaging device for recording the intraprocedural image data may include a magnetic resonance tomography unit (MRT unit) and/or a computed tomography unit (CT unit) and/or a medical X-ray device, in particular a medical C-arm X-ray device, and/or an ultrasound device and/or a positron emission tomography unit (PET unit). The medical imaging device for recording the intraprocedural image data may be identical to or different from the medical imaging device for recording the preprocedural image data.

The intraprocedural image data may advantageously include a, in particular time-resolved, 2D and/or 3D mapping of the examination object, in particular of the hollow organ of the examination object, with the part of the medical object arranged therein, in particular the predefined section. Further, the intraprocedural image data may map the examination object, in particular the hollow organ and the predefined section, intraprocedurally, in particular while the predefined section is arranged in the hollow organ. The intraprocedural dataset may advantageously be registered with a coordinate system of the examination object, in particular a patient coordinate system.

Advantageously, the capturing unit may be configured to identify the instantaneous positioning, in particular the instantaneous spatial position and/or orientation and/or pose, of the predefined section using the intraprocedural image data. Identifying the instantaneous positioning of the predefined section may include identifying, in particular segmenting, a mapping of the predefined section in the intraprocedural image data. Image points, for example, pixels and/or voxels, may be identified in the intraprocedural image data in this connection, which map the predefined section, for example, using a threshold value-based comparison of image values of the image points and/or using geometric features, in particular a marker structure on the predefined section.

The proposed embodiment may enable improved capturing of the instantaneous positioning of the predefined section, in particular in respect of the hollow organ.

In a further advantageous embodiment of the proposed system, moving of the medical object may include a translation and/or rotation.

Advantageously, moving of the medical object by the movement apparatus may include a translation, in particular along a direction of longitudinal extension of the medical object. In the operating state, the movement apparatus may act at the proximal section of the medical object and execute the translation of the medical object, in particular of the predefined section, by advance or withdrawal of the proximal section for this purpose. Further, movement of the medical object by the movement apparatus may include a rotation, in particular about the direction of longitudinal extension of the medical object. In the operating state, the movement apparatus may act at the proximal section of the medical object and execute the rotation of the medical object, in particular of the predefined section, by rotation of the proximal section for this purpose. The movement apparatus may be configured to translate and/or rotate the medical object simultaneously or sequentially. Further, the movement apparatus may be configured to adjust a movement speed and/or a direction of movement of the movement of the medical object, in particular of the predefined section, in particular on the basis of the control specification.

The proposed embodiment may advantageously enable precise robot-assisted positioning and/or movement of the medical object, in particular of the predefined section, inside the hollow organ.

In a further advantageous embodiment of the proposed system, the processing unit may be configured to capture an item of planning information. The item of planning information may specify a target positioning for the predefined section on or in the hollow organ. Further, the processing unit may be configured to provide the control specification additionally on the basis of the target positioning.

The item of planning information may include procedure planning, for example, a tissue, which is to be treated, of the examination object. Capturing the item of planning information may include capturing and/or reading a computer-readable data memory and/or receiving from a data memory unit, for example, a database. Further, the item of planning information may be provided by an input unit for capturing a user input.

The item of planning information may advantageously specify the target positioning, in particular a spatial target position and/or target orientation and/or target pose, for the predefined section. The target positioning may be specified in respect of the hollow organ. Advantageously, the target positioning may be registered with the preprocedural dataset and/or the instantaneous positioning of the predefined section and/or the intraprocedural image data.

The processing unit may advantageously be configured to determine a spatial path, in particular a trajectory and/or a movement path, for the predefined section between its instantaneous positioning and the target positioning, in particular inside the hollow organ, additionally on the basis of the target positioning. Advantageously, the processing unit may also be configured to provide the control specification relating to the specification of the manipulation of the predefined section and/or the movement of the medical object additionally on the basis of the target positioning, in particular the path.

Advantageously, the control specification may have at least one command, in particular a chronological sequence of commands, relating to the specification of a manipulation of the predefined section and/or to the specification of a translation and/or rotation of the medical object, in such a way that the predefined section may be moved along the path to the target positioning.

The proposed embodiment may advantageously enable autonomous, in particular robotic, navigation of interventional instruments to the target positioning.

In a further advantageous embodiment of the proposed system, the processing unit may be configured to identify a planned direction of movement of the predefined section using the item of planning information. Further, the processing unit may be configured to identify a narrowing of the hollow organ along the planned direction of movement of the predefined section using the preprocedural dataset. Further, the processing unit may be configured to provide the control specification in such a way that the predefined section may be moved past the narrowing inside a lumen of the hollow organ in accordance with the control specification.

The processing unit may advantageously be configured to determine a spatial path, in particular a trajectory and/or a movement path, for the predefined section between its instantaneous positioning and the target positioning, in particular inside the hollow organ, additionally on the basis of the target positioning. The processing unit may also be configured to determine a planned direction of movement of the predefined section at its instantaneous positioning, in particular in such a way that the predefined section may be moved from its instantaneous positioning to the target positioning.

Further, the processing unit may be configured to identify a narrowing in the hollow organ along the planned direction of movement of the predefined section using the preprocedural dataset, in particular the representation of the hollow organ in the preprocedural dataset. Identifying the narrowing of the hollow organ may include identifying, in particular segmenting, a representation of the narrowing of the hollow organ in the preprocedural dataset. Data points, for example, pixels and/or voxels, may be identified in the preprocedural dataset in this connection, which represent the obstacle and/or the narrowing, for example, using a threshold-based comparison of data values of the data points and/or using geometric features. The processing unit may advantageously be configured to identify the narrowing of the hollow organ in the planned direction of movement of the predefined section and along a direction of longitudinal extension of the hollow organ.

The narrowing of the hollow organ may include a calcification and/or a medical implant, for example, a stent, and/or indentation of a wall of the hollow organ. The narrowing of the hollow organ may be identified as a section of the hollow organ with, in particular asymmetrical, reduced cross-section, in particular diameter. Advantageously, the narrowing does not close the hollow organ completely, but only partially reduces its cross-section. When a complete closure of the hollow organ is identified along the planned direction of movement of the predefined section, a corresponding workflow indication, in particular a warning, may advantageously be output. In addition, with a complete closure, the movement of the medical object may be adjusted accordingly, for example, stopped.

Advantageously, the processing unit may also be configured to provide the control specification in such a way that the predefined section may move past the narrowing inside a lumen of the hollow organ in accordance with the control specification. For example, in the absence of a narrowing and/or curvature of the hollow organ, the control specification may specify a positioning of the predefined section along a centerline of the hollow organ. With identification of a narrowing along the planned direction of movement of the hollow organ, the control specification may advantageously specify a positioning of the predefined section that deviates from the centerline to enable the predefined section to move past the narrowing inside a remaining lumen of the hollow organ.

The proposed embodiment may advantageously enable improved autonomous, in particular robotic, navigation of the medical instrument, with it being possible to evade narrowings inside the hollow organ.

In a further advantageous embodiment of the proposed system, the manipulation of the predefined section may include a curving, in particular directional curving, of the predefined section.

Advantageously, the medical object may be configured such that the predefined section may be curved, in particular by manipulation, at the proximal section of the medical object, in particular directionally curved. Curving of the predefined section may include length-preserving deforming, in particular bending and/or bowing, of the predefined section along one or more direction(s). In particular, the predefined section may be configured so it may be directionally curved. Curving of the predefined section may be limited to curving, in particular bending and/or bowing, of the predefined section parallel to a predefined plane of curvature. Curving of the predefined section may be characterized by a curvature parameter, in particular a radius of curvature and/or a direction of curvature and/or an angle of curvature. The control specification may specify the curvature parameter for curving of the predefined section.

The proposed embodiment may advantageously enable improved positioning and/or movement of the predefined section inside the hollow organ.

In a further advantageous embodiment of the proposed system, the processing unit may be configured to compare the curvature of the hollow organ at the positioning of the predefined section with a first threshold value and to provide the control specification in such a way that the control specification specifies a curvature for the predefined section, which is larger with respect to the curvature of the hollow organ if the curvature of the hollow organ overshoots the first threshold value. Alternatively, or in addition, the processing unit may be configured to compare the curvature of the hollow organ with a second threshold value and to provide the control specification in such a way that the control specification specifies a curvature for the predefined section, which is smaller with respect to the curvature of the hollow organ, or no curvature, in particular no additional curvature, if the curvature of the hollow organ undershoots the second threshold value. The second threshold value may be less than or equal to the first threshold value.

Advantageously, the processing unit may be configured to characterize the curvature of the hollow organ at the positioning of the predefined section using a curvature parameter of the hollow organ. The curvature parameter of the hollow organ may quantify a radius of curvature and/or a direction of curvature and/or an angle of curvature of the hollow organ at the instantaneous positioning of the predefined section. The comparison of the curvature, in particular of the curvature parameter, of the hollow organ with the first threshold value may include determining a deviation, for example, a difference and/or a quotient, between the curvature parameter of the hollow organ and the first threshold value.

Advantageously, when the first threshold value is overshot by the curvature, in particular the curvature parameter, of the hollow organ at the instantaneous positioning of the predefined section, the control specification may specify a curvature for the predefined section, which is larger with respect to the curvature of the hollow organ, in particular a curvature parameter which is larger with respect to the respective curvature parameter of the hollow organ, for example, a larger radius of curvature and/or angle of curvature.

The comparison of the curvature, in particular of the curvature parameter, of the hollow organ with the second threshold value may include determining a deviation, for example, a difference and/or a quotient, between the curvature parameter of the hollow organ and the second threshold value. In particular, the second threshold value may characterize a smaller curvature of the hollow organ than the first threshold value.

Advantageously, when the second threshold value is undershot by the curvature, in particular the curvature parameter, of the hollow organ at the instantaneous positioning of the predefined section, the control specification may specify a curvature for the predefined section, which is smaller with respect to the curvature of the hollow organ, in particular a curvature parameter that is smaller with respect to the respective curvature parameter of the hollow organ, for example, a smaller radius of curvature and/or angle of curvature. Alternatively, the control specification may specify no curvature, in particular no additional curvature, for the predefined section, if the curvature of the hollow organ undershoots the second threshold value.

The proposed embodiment may advantageously enable positioning and/or movement of the predefined section inside the hollow organ, which extends in a curve, with low risk of injury. In particular, iatrogenic thrombi may advantageously be prevented.

In a further advantageous embodiment of the proposed system, the processing unit may also be configured to identify a diameter of the hollow organ at the positioning of the predefined section using the preprocedural dataset. Further, the processing unit may be configured to compare the diameter with a third threshold value. Furthermore, the processing unit may be configured to provide the control specification in such a way that no curvature, in particular no additional curvature, is specified for the predefined section if the diameter undershoots the third threshold value.

Advantageously, the processing unit may be configured to identify the diameter of the hollow organ at the positioning of the predefined section using the preprocedural dataset, in particular using the representation of the hollow organ, in the preprocedural dataset. Identifying the diameter of the hollow organ at the positioning of the predefined section using the preprocedural dataset may include identifying a cross-sectional area of the hollow organ at the positioning of the predefined section. Further, the processing unit may be configured to compare the diameter of the hollow organ at the positioning of the predefined section with the third threshold value. The third threshold value may advantageously characterize a comparative diameter. The comparison of the diameter of the hollow organ at the positioning of the predefined section with the third threshold value may include determining a deviation, for example, a difference and/or a quotient, between the diameter of the hollow organ at the positioning of the predefined section and the third threshold value.

Advantageously, processing unit the control specification may specify no curvature, in particular no additional curvature, for the predefined section, if the diameter of the hollow organ at the positioning of the predefined section undershoots the third threshold value.

The proposed embodiment may enable improved positioning and/or movement of the predefined section inside the hollow organ. In addition, a length of the medical object introduced into the hollow organ may advantageously be minimized.

In a second aspect, a method for providing a control specification is disclosed. In a first act, a preprocedural dataset is received which has a representation of a hollow organ of the examination object. In a further act, an item of positioning information relating to a positioning of a predefined section of a medical object in the hollow organ of the examination object is received. In a further act, a curvature of the hollow organ at the positioning of the predefined section is identified using the preprocedural dataset and the item of positioning information. In a further act, the control specification is provided on the basis of the curvature of the hollow organ. The control specification has a specification relating to the manipulation of the predefined section.

The advantages of the proposed method correspond to the advantages of the proposed system. Features, advantages, or alternative embodiments mentioned in this connection may likewise also be transferred to the other claimed subject matters, and vice versa.

In a further advantageous embodiment of the proposed method, the manipulation of the predefined section may include a curving, in particular directional curving, of the predefined section.

In a further advantageous embodiment of the proposed method, the curvature of the hollow organ at the positioning of the predefined section may be compared with a first threshold value and the control specification may be provided in such a way that the control specification may specify a curvature for the predefined section, which is larger with respect to the curvature of the hollow organ if the curvature of the hollow organ overshoots the first threshold value. Alternatively, or in addition, the curvature of the hollow organ at the positioning of the predefined section may be compared with a second threshold value and the control specification may be provided in such a way that the control specification may specify a curvature for the predefined section, which is smaller with respect to the curvature of the hollow organ, or no curvature, in particular no additional curvature, if the curvature of the hollow organ undershoots the second threshold value. The second threshold value may be less than or equal to the first threshold value.

In a further advantageous embodiment of the proposed method, a diameter of the hollow organ may be identified at the positioning of the predefined section using the preprocedural dataset. Further, the diameter may be compared with a third threshold value. In addition, the control specification may be provided in such a way that no curvature, in particular no additional curvature, is specified for the predefined section if the diameter undershoots the third threshold value.

In a further advantageous embodiment of the proposed method, an item of planning information may be captured which specifies a target positioning for the predefined section on or in the hollow organ. The control specification may additionally be provided on the basis of the target positioning.

In a further advantageous embodiment of the proposed method, a planned direction of movement of the predefined section may be identified using the item of planning information. Further, an obstacle in the hollow organ and/or a narrowing of the hollow organ along the planned direction of movement of the predefined section may be identified using the preprocedural dataset. The control specification may be provided in such a way that the predefined section may move past the obstacle and/or the narrowing inside a lumen of the hollow organ in accordance with the control specification.

In a third aspect, a computer program product is disclosed, wherein the computer program product includes a computer program that may be loaded directly into a memory of a processing unit, with program segments in order to carry out all acts of a proposed method for providing a control specification when the program segments are executed by the processing unit.

The computer program product may include software with a source code, which still has to be compiled and linked or only has to be interpreted, or an executable software code, which for execution only has to be loaded into the processing unit. The method for providing a control specification by a processing unit may be carried out quickly, in an identically repeatable manner and robustly by way of the computer program product. The computer program product is configured such that it may carry out the method acts by the processing unit.

The computer program product is stored, for example, on a computer-readable storage medium or saved on a network or server from where it may be loaded into the processor of a processing unit, which may be directly connected to the processing unit or may be configured as part of the processing unit. Furthermore, items of control information of the computer program product may be stored on an electronically readable data carrier. The items of control information of the electronically readable data carrier may be configured in such a way that when the data carrier is used in a processing unit, they carry out the method. Examples of electronically readable data carriers are a DVD, a magnetic tape or a USB stick, on which electronically readable items of control information, in particular software, are stored. When these items of control information are read from the data carrier and stored in a processing unit, all embodiments of the previously described methods may be carried out.

An implementation in terms of software has the advantage that even previously used processing units may be upgraded by way of a software update in order to work. Apart from the computer program, such a computer program product may possibly include additional component parts, such as documentation, and/or additional components, as well as hardware components, such as hardware keys (dongles, etc.) use the software.

Embodiments of the disclosure are presented in the drawings and described in more detail below. The same reference numerals are used in different figures for identical features.

### BRIEF DESCRIPTION OF THE DRA WINGS

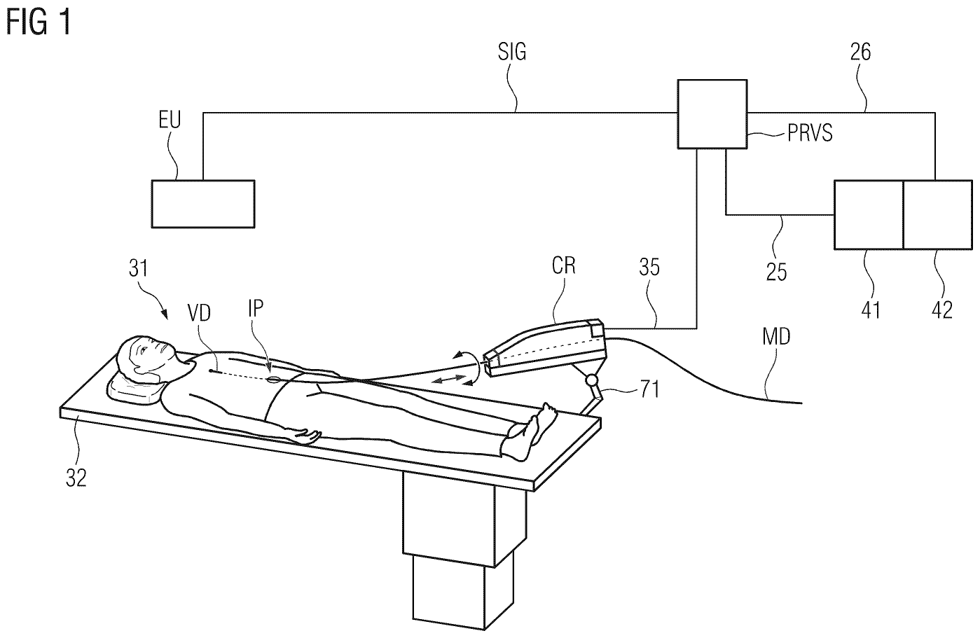

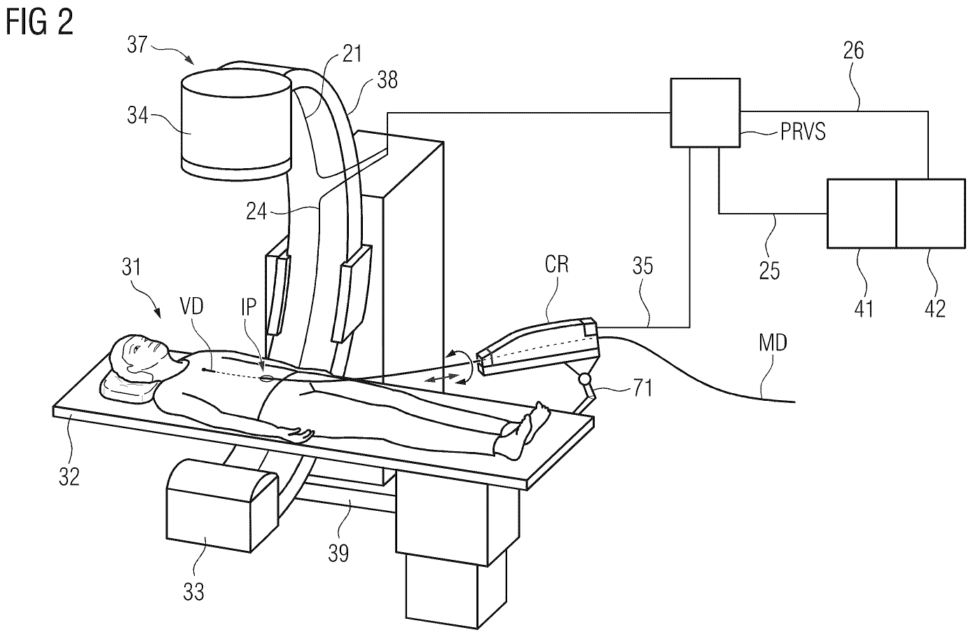

FIGS. 1 and 2 depict schematic presentations of different embodiments of a system for moving a medical object.

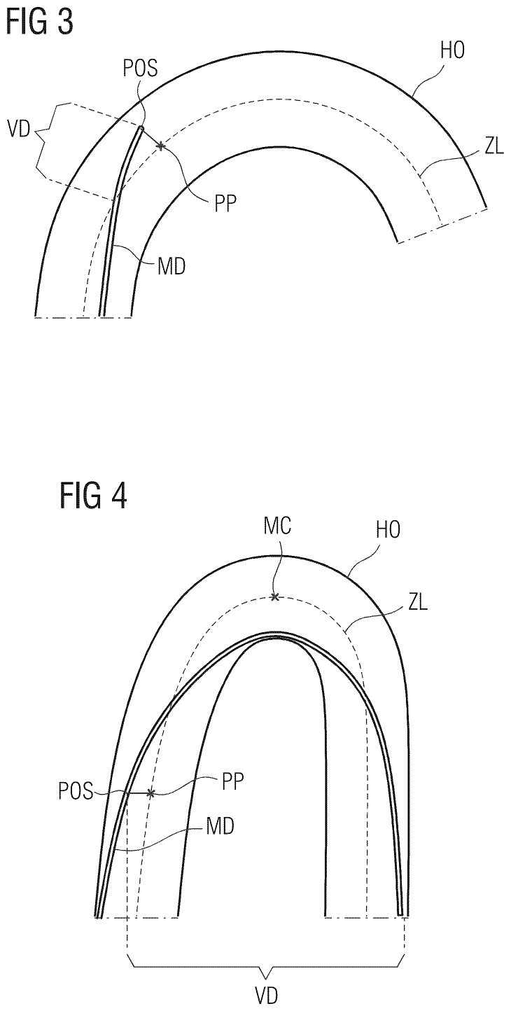

FIGS. 3 to 7 depict schematic presentations of different states of a hollow organ.

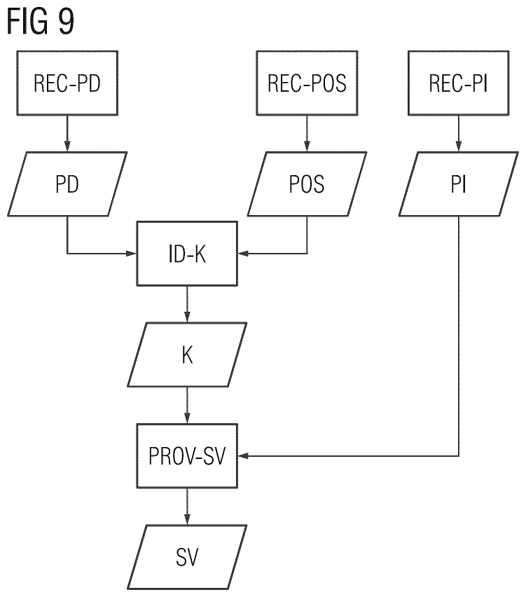

FIGS. 8 and 9 depict schematic presentations of different embodiments of a method for providing a control specification.

### DETAILED DESCRIPTION

FIG. 1 shows a schematic presentation of an advantageous embodiment of a proposed system for moving a medical object MD. The system may include a processing unit PRVS, a movement apparatus CR, and a capturing unit EU. Advantageously, the processing unit PRVS may be configured to receive a preprocedural dataset. The preprocedural dataset may have a representation of a hollow organ of an examination object 31. In an operating state of the system, at least one predefined section VD of the medical object MD may be arranged in the hollow organ of the examination object 31, for example, via an entry port at an entry point IP. The examination object 31 may be arranged on a patient-supporting apparatus 32. The capturing unit EU may be configured to capture an instantaneous positioning of the predefined section VD of the medical object MD. The processing unit PRVS may also be configured to identify a curvature of the hollow organ at the instantaneous positioning of the predefined section VD using the preprocedural dataset. In addition, the processing unit PRVS may be configured to provide the movement apparatus CR with a control specification relating to the movement of the medical object MD on the basis of the curvature of the hollow organ. The control specification may have a specification relating to the manipulation of the predefined section VD. The movement apparatus CR may be configured to move the medical object MD in accordance with the control specification and to manipulate the predefined section VD.

Moving the medical object MD may include a translation and/or rotation. Further, the processing unit PRVS may be configured to capture an item of planning information. The item of planning information may specify a target positioning for the predefined section on or in the hollow organ. The processing unit PRVS may also be configured to provide the control specification additionally on the basis of the target positioning. In addition, the processing unit PRVS may be configured to identify a planned direction of movement of the predefined section VD using the item of planning information. Further, the processing unit PRVS may be configured to identify a narrowing of the hollow organ along the planned direction of movement using the preprocedural dataset. The processing unit PRVS may be configured to provide the control specification in such a way that the predefined section VD may move past the narrowing inside a lumen of the hollow organ in accordance with the control specification.

The manipulation of the predefined section VD may advantageously include a, in particular directional, curving of the predefined section VD.

The movement apparatus CR may be configured, for example, as a catheter robot, in particular for remote manipulation of the medical object MD and of the predefined section VD. Further, the movement apparatus CR may be secured, in particular movably, by a securing element 71, for example, a stand and/or robotic arm, to the patient-supporting apparatus 32. Advantageously, the movement apparatus CR may be configured to move the medical object MD, which in the operating state of the system is arranged at least partially in the movement apparatus CR, in a translational manner at least along a direction of longitudinal extension of the medical object MD. Further, the movement apparatus CR may be configured to rotate the medical object MD around the direction of longitudinal extension. Furthermore, the movement apparatus CR may be configured to manipulate the predefined section VD of the medical object MD, in particular to deform it in a defined manner, for example, via a cable pull inside the medical object MD.

The system may also include an input unit 42, for example, a keyboard, and/or a presentation unit 41, for example, a monitor and/or display. The input unit 42 may be integrated in the presentation unit 41, for example, in the case of a capacitive and/or resistive input display.

The presentation unit 41 may advantageously be configured to display items of information and/or graphical presentations of items of information of the system and/or the capturing unit EU and/or the processing unit PRVS and/or further components. The processing unit PRVS may send, for example, a signal 25 to the presentation unit 41 for this purpose. The input unit 42 may advantageously be configured for capturing a user input and for providing a signal 26 as a function of the user input. Further, the presentation unit 41 may advantageously be configured to display items of information and/or graphical presentations of items of information of the system and/or the processing unit PRVS and/or further components, for example, a graphical presentation of the preprocedural dataset and/or the intraprocedural image data. The processing unit PRVS may send, for example, a signal 25 to the presentation unit 41 for this purpose.

Further, the processing unit PRVS may be configured to provide the movement apparatus CR with the control specification, in particular by the signal 35. The movement apparatus CR is advantageously configured for receiving the control specification using the signal 35. Further, the movement apparatus CR may be configured to move the medical object MD in accordance with the control specification and to manipulate the predefined section VD.

FIG. 2 shows a schematic presentation of a further advantageous embodiment of the proposed system. The capturing unit may have a medical imaging device which is configured to record intraprocedural image data of the examination object 31. Further, the capturing unit may be configured for identifying the instantaneous positioning of the predefined section VD using the intraprocedural image data.

The capturing unit may be configured as a medical imaging device, in particular as a medical C-arm X-ray device 37. The C-arm X-ray device 37 may have a detector 34, in particular an X-ray detector, and an X-ray source 33. To record the intraprocedural image data, the arm 38 of the medical C-arm X-ray device 37 may be mounted to move around one or more axes. Further, the medical C-arm X-ray device 37 may include a movement unit 39, for example, a wheel system and/or rail system and/or a robotic arm, which enables a movement of the medical C-arm X-ray device 37 in the space. The detector 34 and the X-ray source 34 may be secured in a defined arrangement on a shared C-arm 38 so as to move.

The processing unit PRVS may also be configured to control a positioning of the medical C-arm X-ray device 37 relative to the examination object 31 in such a way that the predefined section VD of the medical object MD is mapped in the intraprocedural image data recorded by the medical C-arm X-ray device 37. The positioning of the medical C-arm X-ray device 37 relative to the examination object 31 may include a positioning of the defined arrangement of X-ray source 33 and detector 34, in particular of the C-arm 38, around one or more spatial axes.

To record the intraprocedural image data of the examination object 31, the processing unit PRVS may send a signal 24 to the X-ray source 33. The X-ray source 33 may then emit an X-ray beam bundle, in particular a cone beam and/or fan beam and/or parallel beam. When, after an interaction with the examination region, which is to be mapped, of the examination object 31, the X-ray beam strikes a surface of the detector 34, the detector 34 may send a signal 21 to the processing unit PRVS. The processing unit PRVS may receive the intraprocedural image data, for example, using the signal 21.

FIG. 3 shows a schematic presentation of a first state of a hollow organ. The predefined section VD may be arranged in a lumen of the hollow organ HO. Further, a centerline ZL of the hollow organ is schematically presented in FIG. 3. The instantaneous positioning of the predefined section VD may be determined by a position POS of an end section, in particular a tip, of the medical object MD. Alternatively, the instantaneous positioning of the predefined section VD may be determined as the projection PP of the position POS of the end section onto the centerline ZL of the hollow organ.

FIG. 4 shows a schematic presentation of a further state of a hollow organ. Advantageously, the processing unit PRVS may be configured to compare the curvature of the hollow organ HO at the positioning POS of the predefined section VD with a first threshold value and to provide the control specification in such a way that the control specification specifies a curvature for the predefined section VD, which is greater with respect to the curvature of the hollow organ HO if the curvature of the hollow organ HO overshoots the first threshold value. MC illustrates a point on the centerline ZL of the hollow organ with maximum curvature.

FIG. 5 shows a schematic presentation of a further state of a hollow organ. Advantageously, the processing unit PRVS may be configured to compare the curvature of the hollow organ at the positioning of the predefined section with a second threshold value and to provide the control specification in such a way that the control specification specifies a curvature for the predefined section, which is smaller with respect to the curvature of the hollow organ, or no curvature, in particular no additional curvature, if the curvature of the hollow organ undershoots the second threshold value. The second threshold value may be less than or equal to the first threshold value. Advantageously, the processing unit PRVS may also be configured to identify a diameter D of the hollow organ HO at the positioning of the predefined section VD using the preprocedural dataset. The diameter D of the hollow organ HO may be identified at a cross-sectional area QS of the hollow organ HO at the positioning of the predefined section VD.

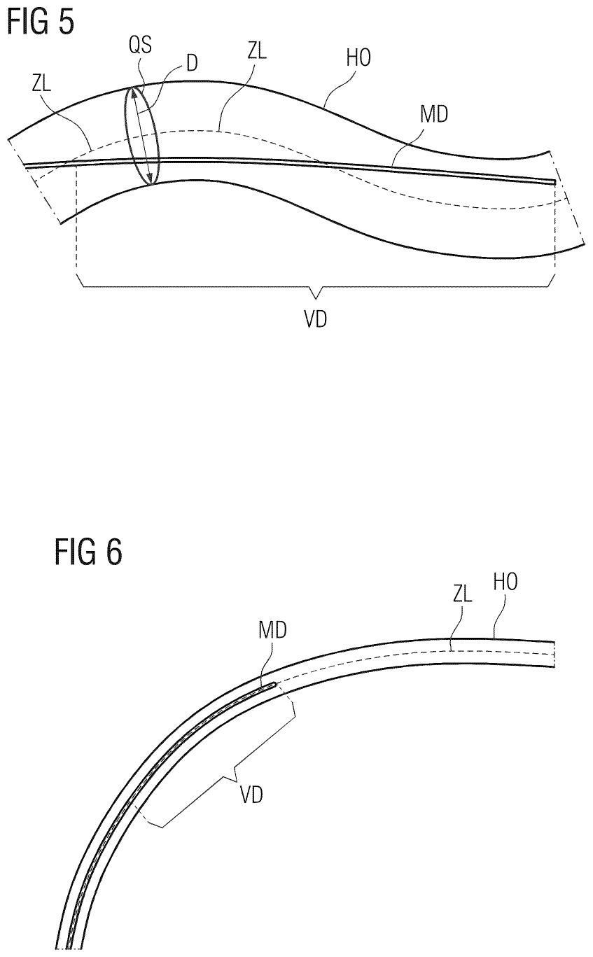

FIG. 6 shows a schematic presentation of a further state of a hollow organ. Advantageously, the processing unit PRVS may also be configured to identify a diameter D of the hollow organ HO at the positioning of the predefined section VD using the preprocedural dataset. Further, the processing unit PRVS may be configured to compare the diameter D with a third threshold value. In addition, the processing unit PRVS may be configured to provide the control specification in such a way that no curvature, in particular no additional curvature, is specified for the predefined section VD if the diameter D undershoots the third threshold value.

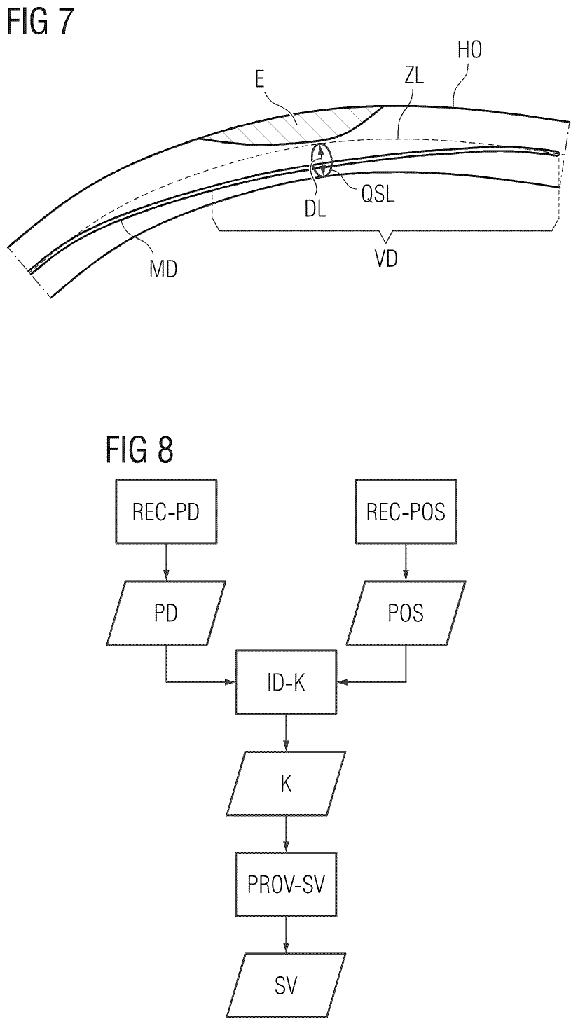

FIG. 7 shows a schematic presentation of a further state of a hollow organ. The processing unit PRVS may be configured to identify a planned direction of movement of the predefined section VD using the item of planning information. Further, the processing unit PRVS may be configured to identify a narrowing E of the hollow organ HO, for example, a calcification, along the planned direction of movement using the preprocedural dataset. The processing unit PRVS may be configured to identify a diameter DL, which is reduced with respect to the rest of the hollow organ HO, of a lumen of the hollow organ HO at the narrowing E, for example, as the diameter D of a cross-section QSL of the lumen at the narrowing E. In addition, the processing unit PRVS may be configured to provide the control specification in such a way that the predefined section VD may move past the narrowing E inside a lumen of the hollow organ HO in accordance with the control specification.

FIG. 8 shows a schematic presentation of an advantageous embodiment of a proposed method for providing a control specification PROV-SV. In a first act, a preprocedural dataset PD may be received REC-PD. The preprocedural dataset PD may have a representation of a hollow organ HO of an examination object 31. In a further act, an item of positioning information POS relating to a positioning of a predefined section VD of a medical object MD in the hollow organ HO of the examination object 31 may be received REC-POS. In a further act, a curvature K of the hollow organ HO at the positioning of the predefined section VD may be identified ID-K using the preprocedural dataset PD and the item of positioning information POS. After this, the control specification SV may be provided PROV-SV on the basis of the curvature K of the hollow organ HO. The control specification SV may have a specification relating to the manipulation of the predefined section VD. Advantageously, the manipulation of the predefined section VD may include directional, curving of the predefined section VD.

Advantageously, the curvature K of the hollow organ HO at the positioning of the predefined section VD may be compared with a first threshold value and the control specification SV may be provided in such a way that the control specification SV specifies a curvature for the predefined section VD, which is larger with respect to the curvature K of the hollow organ HO if the curvature K of the hollow organ HO overshoots the first threshold value. Alternatively or additionally, the curvature K of the hollow organ HO at the positioning of the predefined section VD may be compared with a second threshold value and the control specification SV may be provided in such a way that the control specification SV specifies a curvature for the predefined section VD, which is smaller with respect to the curvature K of the hollow organ HO, or no curvature, in particular no additional curvature, if the curvature K of the hollow organ HO undershoots the second threshold value. The second threshold value may be less than or equal to the first threshold value.

Furthermore, a diameter D of the hollow organ HO may be identified at the positioning of the predefined section VD using the preprocedural dataset PD. The diameter D may be compared with a third threshold value. Further, the control specification SV may be provided in such a way that no curvature, in particular no additional curvature, is specified for the predefined section VD if the diameter D undershoots the third threshold value.

FIG. 9 shows a schematic presentation of a further advantageous embodiment of a proposed method for providing a control specification PROV-SV. An item of planning information PI may be captured REC-PI which specifies a target positioning for the predefined section VD on or in the hollow organ HO. The control specification SV may additionally be provided on the basis of the target positioning.

Advantageously, a planned direction of movement of the predefined section VD may be identified using the item of planning information PI. A narrowing E of the hollow organ HO along the planned direction of movement of the predefined section VD may be identified using the preprocedural dataset PD. In addition, the control specification SV may be provided in such a way that the predefined section VD may move past the narrowing E inside a lumen of the hollow organ HO in accordance with the control specification SV.

The schematic presentations in the described figures are not to scale and do not depict any size ratios.

In conclusion, the methods described in detail above and the presented apparatuses are merely embodiments that may be modified in a wide variety of ways by a person skilled in the art without departing from the scope of the disclosure. Furthermore, use of the indefinite article “a” or “an” does not preclude the relevant features from also being present multiple times. Similarly, the terms “unit” and “element” do not preclude the relevant components from being composed of a plurality of cooperating sub-components which may also be spatially distributed.

In the context of the present application, the expression “on the basis of” may be understood, in particular, within the meaning of the expression “by using.” In particular, wording, according to which a first feature is generated (alternatively: ascertained, determined, etc.) on the basis of a second feature, does not preclude the first feature from being generated (alternatively: ascertained, determined, etc.) on the basis of a third feature.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A system for moving a medical object, the system comprising:
a processing unit;
a movement apparatus; and
a capturing unit,
wherein the processing unit is configured to receive a preprocedural dataset that has a representation of a hollow organ of an examination object,
wherein a predefined section of the medical object is arranged in the hollow organ of the examination object in an operating state of the system,
wherein the capturing unit is configured to capture an instantaneous positioning of the predefined section of the medical object,
wherein the processing unit is configured to identify a curvature of the hollow organ at the instantaneous positioning of the predefined section using the preprocedural dataset,
wherein the processing unit is configured to provide the movement apparatus with a control specification for movement of the medical object based on the curvature of the hollow organ,
wherein the control specification has a specification relating to the manipulation of the predefined section, and
wherein the movement apparatus is configured to move the medical object in accordance with the control specification and manipulate the predefined section.

2. The system of claim 1, wherein the capturing unit has a medical imaging device configured to record intraprocedural image data of the examination object, and
wherein the capturing unit is configured to identify the instantaneous positioning of the predefined section using the intraprocedural image data.

3. The system of claim 1, wherein the movement of the medical object comprises a translation and/or rotation.

4. The system of claim 1, wherein the processing unit is further configured to:
capture an item of planning information that specifies a target positioning for the predefined section on the hollow organ or in the hollow organ; and
provide the control specification additionally based on the target positioning.

5. The system of claim 4, wherein the processing unit is further configured to:
identify a planned direction of movement of the predefined section using the item of planning information;
identify a narrowing of the hollow organ along the planned direction of movement using the preprocedural dataset; and
provide the control specification such that the predefined section is configured to move past the narrowing inside a lumen of the hollow organ in accordance with the control specification.

6. The system of claim 1, wherein the manipulation of the predefined section comprises a directional curving of the predefined section.

7. The system of claim 6, wherein the processing unit is further configured to:
compare the curvature of the hollow organ at the positioning of the predefined section with a first threshold value and provide the control specification such that the control specification specifies a curvature for the predefined section that is larger with respect to the curvature of the hollow organ when the curvature of the hollow organ overshoots the first threshold value; and/or
compare the curvature of the hollow organ at the positioning of the predefined section with a second threshold value and provide the control specification such that the control specification specifies: (i) a curvature for the predefined section that is smaller with respect to the curvature of the hollow organ, or (ii) no additional curvature, when the curvature of the hollow organ undershoots the second threshold value,
wherein the second threshold value is less than or equal to the first threshold value.

8. The system of claim 7, wherein the processing unit is further configured to:
identify a diameter of the hollow organ at the positioning of the predefined section using the preprocedural dataset;
compare the diameter with a third threshold value; and
provide the control specification such that no additional curvature is specified for the predefined section when the diameter undershoots the third threshold value.

9. The system of claim 1, wherein the processing unit is further configured to:
identify a diameter of the hollow organ at the positioning of the predefined section using the preprocedural dataset;
compare the diameter with a threshold value; and
provide the control specification such that no additional curvature is specified for the predefined section when the diameter undershoots the threshold value.

10. A method for providing a control specification, the method comprising:
receiving a preprocedural dataset having a representation of a hollow organ of an examination object;
receiving an item of positioning information relating to a positioning of a predefined section of a medical object in the hollow organ of the examination object;
identifying a curvature of the hollow organ at the positioning of the predefined section using the preprocedural dataset and the item of positioning information; and
providing the control specification based on the curvature of the hollow organ, wherein the control specification has a specification relating to a manipulation of the predefined section.

11. The method of claim 10, wherein the manipulation of the predefined section comprises a directional curving of the predefined section.

12. The method of claim 11, wherein the curvature of the hollow organ at the positioning of the predefined section is compared with a first threshold value and the control specification is provided such that the control specification specifies a curvature for the predefined section that is larger with respect to the curvature of the hollow organ when the curvature of the hollow organ overshoots the first threshold value, and/or
wherein the curvature of the hollow organ at the positioning of the predefined section is compared with a second threshold value and the control specification is provided such that the control specification specifies: (i) a curvature for the predefined section that is smaller with respect to the curvature of the hollow organ, or (ii) no additional curvature, when the curvature of the hollow organ undershoots the second threshold value,
wherein the second threshold value is less than or equal to the first threshold value.

13. The method of claim 12, wherein a diameter of the hollow organ is identified using the preprocedural dataset at the positioning of the predefined section,
wherein the diameter is compared with a third threshold value, and
wherein the control specification is provided such that no additional curvature is specified for the predefined section when the diameter undershoots the third threshold value.

14. The method of claim 10, wherein a diameter of the hollow organ is identified using the preprocedural dataset at the positioning of the predefined section,
wherein the diameter is compared with a threshold value, and
wherein the control specification is provided such that no additional curvature is specified for the predefined section when the diameter undershoots the threshold value.

15. The method of claim 10, wherein an item of planning information is captured that specifies a target positioning for the predefined section on the hollow organ or in the hollow organ, and
wherein the control specification is provided additionally based on the target positioning.

16. The method of claim 15, wherein a planned direction of movement of the predefined section is identified using the item of planning information,
wherein a narrowing of the hollow organ along the planned direction of movement of the predefined section is identified using the preprocedural dataset, and
wherein the control specification is provided such that the predefined section is moved past the narrowing inside a lumen of the hollow organ in accordance with the control specification.

17. A computer program product having a computer program configured to be loaded directly into a memory of a processing unit, wherein the computer program, when executed by the processing unit, is configured to:
receive a preprocedural dataset having a representation of a hollow organ of an examination object;
receive an item of positioning information relating to a positioning of a predefined section of a medical object in the hollow organ of the examination object;
identify a curvature of the hollow organ at the positioning of the predefined section using the preprocedural dataset and the item of positioning information; and
provide the control specification based on the curvature of the hollow organ, wherein the control specification has a specification relating to a manipulation of the predefined section.

18. The system of claim 1, wherein the manipulation of the predefined section comprises a curving of the predefined section.

19. The method of claim 10, wherein the manipulation of the predefined section comprises a curving of the predefined section.
